(19) United States
(12) Patent Application Publication
(10) Pub. No.: US 2008/0114183 A1
(43) Pub. Date: May 15, 2008

(54) Monomers comprising superacidic groups, and polymers therefrom

(75) Inventors: David Roger Moore, Albany, NY (US); Hongyi Zhou, Niskayuna, NY (US); Daniel Joseph Brunelle, Burnt Hills, NY (US); Joyce Hung, Niskayuna, NY (US); Hongwei Liu, Troy, NY (US); Daniel Steiger, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 11/598,947

(22) Filed: Nov. 14, 2006

(51) Int. Cl. C07C 309/19; C07C 309/65

(52) U.S. Cl. 558/51; 562/42

(57) ABSTRACT

The invention relates generally to monomers comprising superacidic functional groups. The superacidic functional groups comprise fluorinated sulfonate moieties. Monomers provided by the present invention include dihydroxy aromatic compounds, aromatic diamines, aromatic dicarboxylic acids, aromatic diacarboxylic acid esters, aromatic dithiols, and monomers comprising mixed functionalities, for example monomers comprising an aromatic amine group and an aromatic hydroxyl group. The monomers provided by the present invention are useful in the preparation of novel polymers comprising superacidic functional groups, materials useful in membrane applications. The superacidic functional groups present in the polymer compositions impart excellent proton conductivities. In one embodiment, the present invention provides monomers which may be used to prepare polymers useful as materials for polymer electrolyte fuel cell membranes.

### BACKGROUND

The invention relates generally to compositions comprising superacidic functional groups. In one embodiment, the present invention relates to compositions comprising perfluorosulfonate moieties. In a further embodiment, the present invention relates to polymer compositions comprising superacidic functional groups.

Interest in using fuel cells as a clean, alternative power source has spurred intense research in polymer electrolyte membrane (PEM) fuel cell development to meet the cost and performance requirements for automotive and portable applications. Current PEM fuel cells use mainly Nafion® and/or other perfluorosulfonic acid polymer membranes which have high proton conductivity and good chemical and mechanical stability at high relative humidity. Notwithstanding the availability of known perfluorosulfonic acid polymer membranes such as the Nafion® based systems, there remains a need for further improvements in membrane performance under certain conditions of use, for example use at low relative humidity. Therefore, alternative membrane materials displaying enhanced performance characteristics relative to known materials are desired. In particular, there is a need to provide highly proton conducting polymeric materials displaying excellent chemical and thermal stability, robust film-forming properties, and which are soluble in common solvents.

### BRIEF DESCRIPTION

In one embodiment, the present invention provides a monomer having formula I

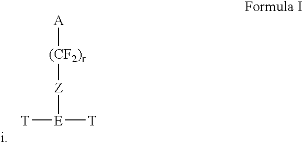

wherein E is a C₅-C₅₀ aromatic radical;

### Z is a bond, O, S, SO, SO₂, a C₁-C₂₀ aliphatic radical, a C₃-C₄₀ aromatic radical, or a C₄-C₂₀ cycloaliphatic radical;

“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol; and

“r” is an integer ranging from 1 to 20.

In another embodiment, the invention provides a monomer having formula V

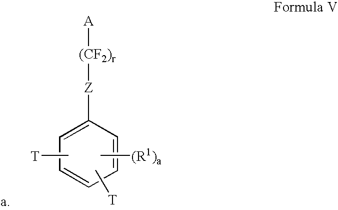

wherein Z is a bond, O, S, SO, SO₂, a C₁-C₂₀ aliphatic radical, a C₃-C₄₀ aromatic radical, or a C₄-C₂₀ cycloaliphatic radical;
“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol;

### R¹ is a C₁-C₄₀ aliphatic radical, a C₃-C₄₀ aromatic radical, or a C₄-C₂₀ cycloaliphatic radical;

“r” is an integer ranging from 1 to 20; and
“a” is 0 or an integer ranging from 1 to 3.

In a further embodiment, the invention provides a monomer having formula VII

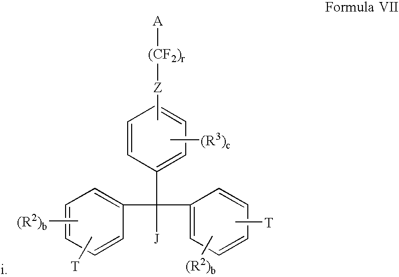

wherein J is a hydrogen, a C₁-C₂₀ aliphatic radical, a C₃-C₂₀ aromatic radical, or a C₄-C₂₀ cycloaliphatic radical;

### Z is a bond, O, S, SO, SO₂, a C₁-C₂₀ aliphatic radical, a C₃-C₄₀ aromatic radical, or a C₄-C₂₀ cycloaliphatic radical;

“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol;

### R² and R³ are independently at each occurrence a C₁-C₂₀ aliphatic radical, a C₃-C₄₀ aromatic radical, or a C₄-C₂₀ cycloaliphatic radical;

“r” is an integer ranging from 1 to 20;
“b” is 0 or an integer ranging from 1 to 4; and
“c” is 0 or an integer ranging from 1 to 4.

### DETAILED DESCRIPTION

The singular forms “a”, “an” and “the” include plural referents unless the context clearly dictates otherwise.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph—), 4-(3-bromoprop-1-yl)phen-1-yl (i.e., 4-BrCH₂CH₂CH₂Ph—), and the like. Further examples of aromatic radicals include 4-allyloxyphen-1-oxy, 4-aminophen-1-yl (i.e., 4-H₂NPh—), 3-aminocarbonylphen-1-yl (i.e., NH₂COPh—), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph—), 4-mercaptomethylphen-1-yl (i.e., 4-HSCH₂Ph—), 4-methylthiophen-1-yl (i.e., 4-CH₃SPh—), 3-methoxyphen-1-yl, 2-methoxycarbonylphen-1-yloxy (e.g., methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is a cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂ C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g., CH₃CHBrCH₂C₆H₁₀O—), and the like. Further examples of cycloaliphatic radicals include 4-allyloxycyclohex-1-yl, 4-aminocyclohex-1-yl (i.e., H₂C₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy (2-CH₃OCOC₆H₁₀—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g., (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilylpropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

As noted, the present invention relates to compositions comprising superacidic functional groups. As used herein, the term superacidic functional group refers to organic fluorosulfonic acid groups (e.g. —CF₂SO₃H), salts of organic fluorosulfonic acid groups (e.g. —CF₂CF₂CF₂SO₃⁻NH₄⁺), and derivatives of organic fluorosulfonic acid groups which upon exposure to water liberate organic fluorosulfonic acid groups (e.g. —CF₂CF₂CF₂SO₂F gives CF₂CF₂CF₂SO₃H upon hydrolysis). In general, the organic fluorosulfonic acid groups typically comprise covalently bound fluorine atoms in close proximity to a sulfonic acid moiety. In one embodiment, the superacidic functional group is a polyfluorosulfonate group, for example a perfluoro ethylene group (—CF₂CF₂—) covalently linked at one end to a sulfonic acid (—SO₃H), a salt of a sulfonic acid (e.g. (—SO₃Li)), or a sulfonate ester (e.g. (—SO₃Ph)). In particular embodiments, the superacidic functional group is a perfluoro oxyethylene group (—CF₂CF₂OCF₂CF₂—) group covalently linked at one end to a sulfonic acid (—SO₃H), a salt of a sulfonic acid, or a sulfonate ester. In one embodiment, the present invention provides a monomer species comprising at least one superacidic functional group. In one embodiment, the monomer may be represented generically by formula I

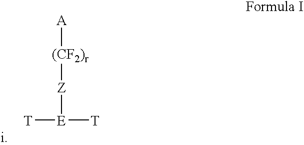

wherein E is a C₅-C₅₀ aromatic radical;

### Z is a bond, O, S, SO, SO₂, a C₁-C₂₀ aliphatic radical, a C₃-C₄₀ aromatic radical, or a C₄-C₂₀ cycloaliphatic radical;

“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol; and

“r” is an integer ranging from 1 to 20.

In the monomer represented by formula I, the group —(CF₂)ᵣ-A represents a superacidic functional group. Monomers having formula I comprising superacidic functional groups are exemplified in Table 1. The exemplary monomers 1a-1m in Table 1 illustrate specific embodiments of the genus defined by formula I.

TABLE 1
Exemplary monomers having formula I

| Entry | | | Z | Value | A | T |
|---|---|---|---|---|---|---|
| # | Monomers | E Group | Group | of “r” | Group | Group |
| 1a | 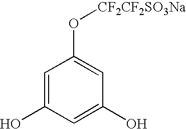 | C₆AromaticRadical | O | 2 | SO₃Na | OH |
| 1b | 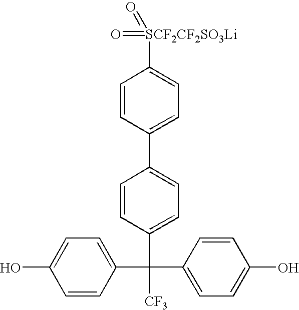 | C₂₆AromaticRadical | SO₂ | 2 | SO₃Li | OH |
| 1c | 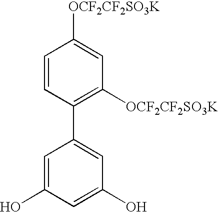 | C₁₄AromaticRadical | O | 2 | SO₃K | OH |
| 1d | 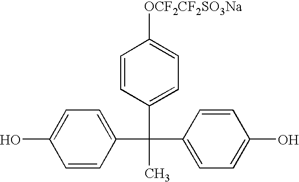 | C₂₀AromaticRadical | O | 2 | SO₃Na | OH |
| 1e | 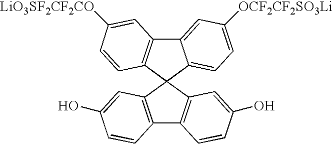 | C₂₇AromaticRadical | O | 2 | SO₃Li | OH |
| 1f | 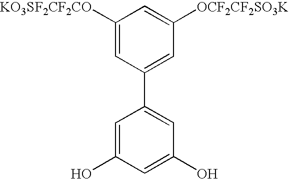 | C₁₄AromaticRadical | O | 2 | SO₃K | OH |
| 1g | 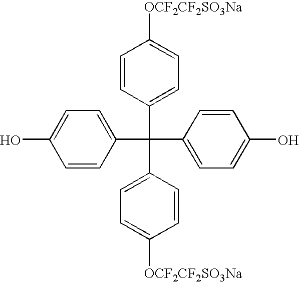 | C₂₇AromaticRadical | O | 2 | SO₃Na | OH |
| 1h | 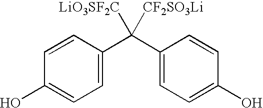 | C₁₄AromaticRadical | Bond | 1 | SO₃Li | OH |
| 1i | 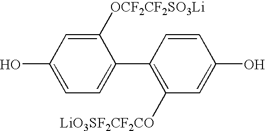 | C₁₄AromaticRadical | O | 2 | SO₃Li | OH |
| 1j | 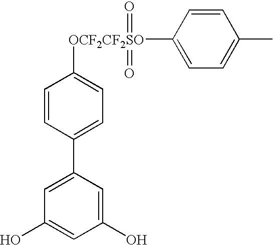 | C₁₂AromaticRadical | O | 2 | 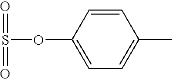 | OH |
| 1k | 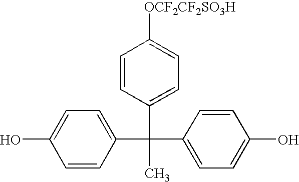 | C₃₀AromaticRadical | O | 2 | SO₃H | OH |
| 1l | 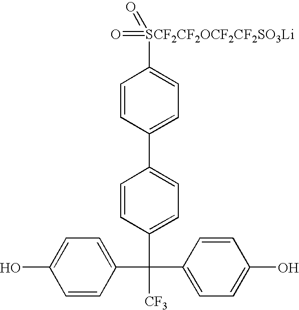 | C₂₆AromaticRadical | 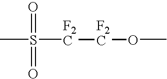 | 2 | SO₃Li | OH |
| 1m | 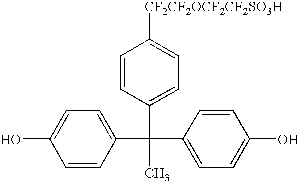 | C₂₀AromaticRadical | CF₂CF₂O | 2 | SO3H | OH |

The monomer of Entry-1a represents a resorcinol-like monomer comprising a superacidic functional group wherein “E” in formula I is a C₆ aromatic radical having formula II

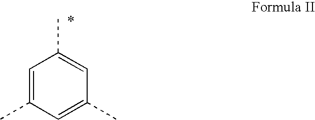

wherein the dashed line [symbol] signals the point of attachment of the group Z, while the dashed lines [symbol] signal the point of attachment of the T groups, Z is an oxygen atom, “r” is 2, the group “A” is the sodium salt of a sulfonic acid, and the T groups are each hydroxyl. The monomer of Entry-1b represents a bisphenol-like monomer comprising a superacidic functional group wherein “E” in formula I is a C₂₆ aromatic radical having formula III

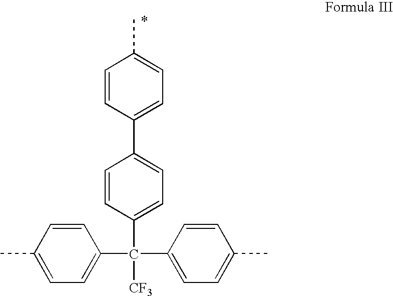

wherein the dashed line [symbol] signals the point of attachment of the group Z, while the dashed lines [symbol] signal the point of attachment of the T groups, Z is a sulfonyl (SO₂) group, “r” is 2, the group “A” is the lithium salt of a sulfonic acid, and the T groups are each hydroxyl. The monomer of Entry-1e represents a spirobifluorene-like monomer comprising two superacidic functional groups wherein “E” in formula I is a C₂₇ aromatic radical having formula IV

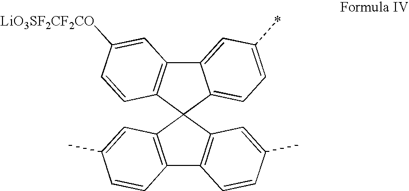

wherein the dashed line -----* signals the point of attachment of the group Z, while the dashed lines ------ signal the point of attachment of the T groups, Z is an oxygen atom, “r” is 2, the group “A” is the lithium salt of a sulfonic acid, and the T groups are each hydroxyl. With respect to the relationship between generic formula I and the species represented by Entry-1e of Table 1, those skilled in the art will appreciate that the group “E” of formula I corresponds to a C₂₇ aromatic radical which comprises one of the two substructures —OCF₂CF₂SO₃Li present. It should be noted that, as defined herein, an aromatic radical may comprise a wide variety of functional groups and/or heteroatoms. Consonant with the definition provided herein of the term “aromatic radical”, a radical is deemed to be an aromatic radical when the group of atoms being referred to meets the threshold requirement that it comprises at least one aromatic group (i.e. it comprises at least one aromatic ring). The monomer of Entry-1l represents yet another monomer of the present invention wherein the Z group in formula I is a (SO₂CF₂CF₂O) moiety.

As noted, in one embodiment, the present invention provides a class of novel monomers having general formula I wherein the E group may comprise a wide variety of functional groups. These functionalities, which are in addition to those represented by the T groups, the Z group and the superacidic functional group (CF₂)ᵣA, may provide the monomer with other desirable properties that may be required in various applications. Some exemplary properties include increased acidity, reactive sites for functionalization and crosslinking, improved solubility, compatibility, and the like. A useful principle is that greater acidity of the monomer will make the polymer derived from said monomer more acidic, thus enhancing the proton exchange capabilities of the polymer, giving rise to higher proton conductivity values. Reactive sites for functionalization may be used to provide other functional groups on the polymer to give other desired properties. Alternately, the functional groups may be used to react with other compounds to provide pendant units. Some useful pendant units include, but are not limited to, long chain aliphatic units which may promote liquid crystalline behavior, short chain aliphatic, aromatic or cycloaliphatic units to improve solubility, aromatic units to increase glass transition temperature, and so on. Functional groups comprised within the group E of a monomer having formula I may be used to effect crosslinking of a polymer derived from said monomer. As is understood by those skilled in the art, crosslinking may be effected to impart good recovery properties, and/or to impart high rigidity and dimensional stability in a variety of polymer systems. In some instances, a polymer initially having a relatively low glass transition temperature is desired, so that the polymer may be shaped into an article at relatively low temperatures. This feature is of value when preparing articles comprising polymers derived from monomers of the present invention comprising superacidic functional groups. In one embodiment, a polymer comprising structural units derived from a monomer of the present invention further comprises functional groups which may be used to effect crosslinking at a temperature slightly higher than the temperature needed to shape the polymer into an article. Thus, the polymer may be shaped into a first article at a lower first temperature, and subsequently the polymer may be crosslinked at a higher second temperature to provide a second article exhibiting higher dimensional stability than said first article. Thus in one embodiment, an appropriately functionalized monomer having formula I is polymerized, shaped into an article, and subsequently, the shaped article is subjected to a crosslinking step.

The organic solubility of monomers having formula I and polymers derived from them may be enhanced through the inclusion of pendant organic substituents (for example octyl groups) comprised within group E that tend to render the monomer and polymers derived from the monomer more soluble in organic solvents. The water solubility of monomers having formula I and polymers derived from them may be enhanced through the inclusion of polar substituents (for example carboxylate groups) comprised within group E that tend to render the monomer and polymers derived from the monomer more soluble in water. Enhanced polymer solubility is desirable in a variety of applications, for example in the preparation of solvent cast films useful as polymer electrolyte membranes.

The monomer represented by formula I comprises a substructure (CF₂)ᵣ which may at times herein be referred to as a perfluoroalkylene group. Without wishing to be bound by any theory, the (CF₂)ᵣ unit is understood to increase the acidity of an sulfonic acid moiety (SO₃H) in proximity to it.

In various embodiments, the present invention provides monomers comprising one or more sulfonate moieties designated “A” groups, wherein “A” is a sulfonate moiety selected from the group consisting of a sulfonic acid moiety, a salt of a sulfonic acid moiety having formula SO₃M, and a sulfonate ester moiety having formula SO₃R, wherein M is an inorganic cation, an organic cation or a mixture thereof, and R is a C₁-C₂₀ aliphatic radical, a C₃-C₂₀ aromatic radical, or a C₄-C₂₀ cycloaliphatic radical. In some embodiments, when “A” is a salt of a sulfonic acid moiety having formula SO₃M, wherein M is an inorganic cation. Exemplary inorganic cations include, but are not limited to, group I metal cations such as cations of sodium, lithium, cesium, and the like; group II metal cations such as cations of calcium, magnesium, and the like; group III metal cations such as cations of aluminum, gallium and the like; transition metal cations such as cations of iron, copper, cobalt, zinc, scandium, titanium, manganese, tungsten, and the like; and inorganic ammonium cations such as NH₄⁺, ND₄⁺ and NT₄⁺. In some specific embodiments, when M is a metal cation, it is selected from the group consisting of cations of potassium, sodium, lithium, and cesium. In one embodiment, M is an organic cation, for example an organic ammonium cation (e.g., tetraalkyl ammonium, hexaalkyl guanidinium, and N-alkyl imidazolium) or an organic phosphonium cation (e.g. tetraphenylphosphonium, methyltriphenylphosphonium, and methyltributylphosphonium). In other embodiments, “A” is a sulfonate ester moiety having formula SO₃R, wherein R is as defined as in formula I. Suitable sulfonate esters are exemplified by p-tolyl sulfonate ester (R is a C₇ aromatic radical), benzyl sulfonate ester (R is a C₇ aromatic radical), methyl sulfonate ester (R is a C₁ aliphatic radical), methyl cyclohexyl sulfonate ester (R is a C₇ cycloaliphatic radical), and t-butyl sulfonate ester (R is a C₄ aliphatic radical). Monomers comprising sulfonate ester groups may be prepared using standard organic chemical techniques from, for example the corresponding monomer comprising a sulfonyl halide group, for example a monomer comprising a sulfonyl chloride group or a sulfonyl fluoride group.

As will be understood by those skilled in the art, formula I embraces a wide variety of monomers which may be converted into polymers comprising superacidic functional groups. In one embodiment, the present invention provides a monomer having formula V

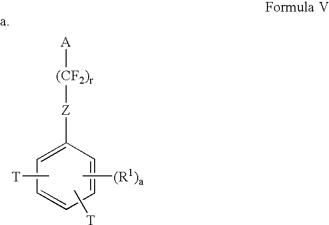

wherein Z is a bond, O, S, SO, SO₂, a C₁-C₂₀ aliphatic radical, a C₃-C₄₀ aromatic radical, or a C₄-C₂₀ cycloaliphatic radical;

### “A” is a sulfonate moiety selected from the group consisting of a sulfonic acid moiety, a salt of a sulfonic acid moiety having formula SO₃M, and a sulfonate ester moiety having formula SO₃R; wherein M is an inorganic cation or an organic cation;

### R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol;

### R¹ is a C₁-C₄₀ aliphatic radical, a C₃-C₄₀ aromatic radical, or a C₄-C₂₀ cycloaliphatic radical;

“r” is an integer ranging from 1 to 20; and
“a” is 0 or an integer ranging from 1 to 3.

Those skilled in the art will recognize that formula V may in certain embodiments represent a subgenus of formula I wherein E is a substituted phenyl group, comprising “a” R¹ groups where “a” is 0 or an integer ranging from 1 to 3 wherein the total number of carbons attributable to the substituted phenyl group and the “a” R¹ groups is from 5 carbons to 50 carbons. Put another way, the monomer having formula V represents a subgenus of the monomer having formula I when the total number of carbon atoms present in the monomer of formula V not attributable to the T groups, the Z group, the (CF₂)ᵣ group or the “A” group, is from 5 carbons to 50 carbons. Monomers of the present invention exemplifying formula V as a subgenus of formula I are exemplified in Table 1 by Entry-1a and in Table 2 by Entry-2a, Entry-2b, Entry-2c, Entry-2d, and Entry-2e. Entry-2f exemplifies a monomer encompassed by generic formula V that is not encompassed by generic formula I, because the total number of carbon atoms present in the monomer of Entry-2f (formula V) not attributable to the T groups, the Z group, the (CF₂)ᵣ group or the “A” group, falls outside of the range from 5 carbons to 50 carbons. The total number of carbon atoms present in the monomer of Entry-2f not attributable to the T groups, the Z group, the (CF₂)ᵣ group or the “A” group, is 54 carbon atoms, i.e. the carbon atoms attributable to the phenyl ring (six carbons) plus the 48 carbon atoms attributable to the two substituents R¹, wherein R¹ represents the C₂₋₄ alkyl group, (CH₂)₂₃CH₃.

TABLE 2
Exemplary monomers having formula V

| Entry | | Z | Value | A | T | | |
|---|---|---|---|---|---|---|---|
| # | Monomers | Group | of “r” | Group | Group | R¹ | “a” |
| 2a | 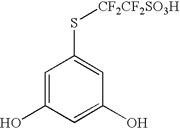 | S | 2 | SO₃H | OH | — | 0 |
| 2b | 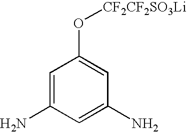 | O | 2 | SO₃Li | NH₂ | — | 0 |
| 2c | 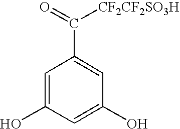 | CO | 2 | SO₃H | OH | — | 0 |
| 2d | 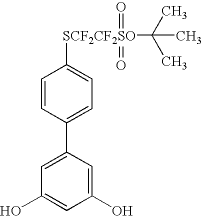 | 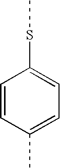 | 2 | 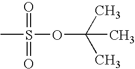 | OH | — | 0 |
| 2e | 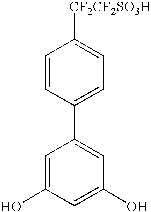 | 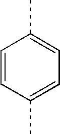 | 2 | SO₃H | OH | — | 0 |
| 2f | 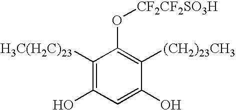 | O | 2 | SO₃H | OH | (CH₂)₂₃—CH₃ | 2 |

Among monomers encompassed by formula V, when both of the T groups are hydroxyl groups (as in Entries-2a, c, d, e and f) the monomer may be regarded as a derivative of a dihydroxy benzene, for example a derivative of 1,3-resorcinol. When both of the T groups are amino groups (e.g. —NH₂) as in Entry-2b or protonated amino groups (e.g. —NH₃₊), the monomer may be regarded as a derivative of a diamino benzene, for example a derivative of meta-phenylene diamine, para-phenylene diamine or ortho-phenylene diamine.

In a specific embodiment, the present invention provides a monomer having formula VI.

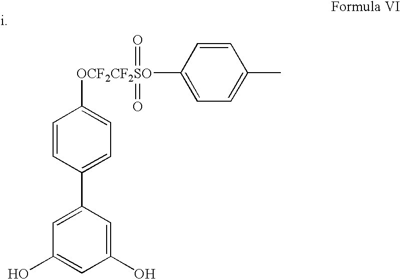

In another embodiment, the present invention provides a monomer comprising formula VII

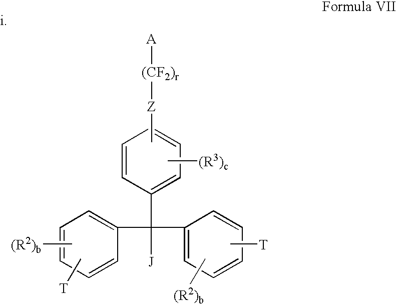

wherein J is a hydrogen, a C₁-C₂₀ aliphatic radical, a C₃-C₂₀ aromatic radical, or a C₄-C₂₀ cycloaliphatic radical;

### Z is a bond, O, S, SO, SO₂, a C₁-C₂₀ aliphatic radical, a C₃-C₄₀ aromatic radical, or a C₄-C₂₀ cycloaliphatic radical;

### “A” is a sulfonate moiety selected from the group consisting of a sulfonic acid moiety, a salt of a sulfonic acid moiety having formula SO₃M, and a sulfonate ester moiety having formula SO₃R; wherein M is an inorganic cation, or an organic cation;

### R is a C₁-C₂₀ aliphatic radical, a C₃-C₂₀ aromatic radical, or a C₄-C₂₀ cycloaliphatic radical;

### T is a functional group selected from the group consisting of hydroxyl, amine, carboxylic acid, carboxylic acid ester, and thiol;

### R² and R³ are independently a C₁-C₂₀ aliphatic radical, a C₃-C₄₀ aromatic radical, or a C₄-C₂₀ cycloaliphatic radical;

“r” is an integer ranging from 1 to 20;
“b” is 0 or an integer ranging from 1 to 4; and
“c” is 0 or an integer ranging from 1 to 4.

Those skilled in the art will understand that formula VII may in certain embodiments represent a subgenus of formula I wherein E is an aromatic radical comprising a triphenylmethyl group and a group J, the triphenylmethyl group comprising 2×“b” R² groups and “c” R³ groups, wherein “b” is 0 or an integer from 1 to 4, and wherein “c” is 0 or an integer from 1 to 4, wherein the total number of carbons attributable to the triphenylmethyl group, the J group, the 2×“b” R² groups, and the “c” R³ groups is from 5 carbons to 50 carbons. Put another way, the monomer having formula VII represents a subgenus of the monomer having formula I when the total number of carbon atoms present in the monomer of formula VII not attributable to the T groups, the Z group, the (CF₂)ᵣ group or the “A” group, is from 5 carbons to 50 carbons. Monomers of the present invention exemplifying formula VII as a subgenus of formula I are exemplified in Table 1 by Entry-1b, Entry-1d, Entry-1g, Entry-1k, Entry-1l, and Entry-1m, and in Table 3 by Entry-3a, Entry-3b, and Entry-3c. Entry-3d exemplifies a monomer encompassed by generic formula VII that is not encompassed by generic formula I, because the total number of carbon atoms present in the monomer of Entry-3d (formula VII) not attributable to the T groups, the Z group, the (CF₂)ᵣ group or the “A” group, falls outside of the range from 5 carbons to 50 carbons. The total number of carbon atoms present in the monomer of Entry-3d not attributable to the T groups, the Z group, the (CF₂)ᵣ group or the “A” group, is 60 carbon atoms, i.e. the 19 carbon atoms attributable to the triphenylmethyl group plus the 40 carbon atoms attributable to the two substituents R², wherein R² represents the C₂₀ alkyl group, —(CH₂)₁₉CH₃, plus the 1 carbon atom attributable to the J group, CF₃.

TABLE 3
Exemplary monomers having formula VII

| | | | | | | | |
|---|---|---|---|---|---|---|---|
| Entry | | J | Z | A | T | | |
| # | Monomer VII | Group | Group | Group | Group | R², R³ | a, b |

| | | | | | | | |
|---|---|---|---|---|---|---|---|
| 3a | 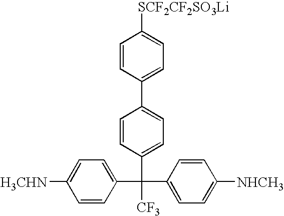 | CF₃ | S | SO₃Li | NHMe | —, — | 0, 0 |
| 3b | 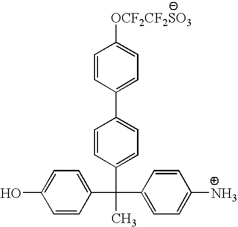 | CH₃ | O | SO₃⁻ | OH, NH₃+ | —, — | 0, 0 |
| 3c | 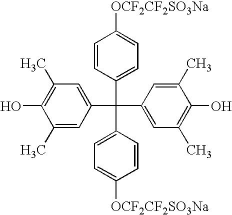 | 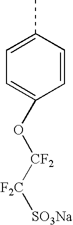 | O | SO₃Na | OH | CH₃, — | 2, 0 |
| 3d | 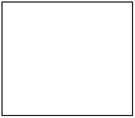 | CF₃ | O | SO₃Li | NH₂ | (CH₂)₁₉CH₃, — | 1, 0 |
| 3e | 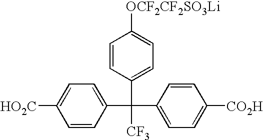 | CF₃ | O | SO₃Li | CO₂H | —, — | 0, 0 |

In each of Entries 3a–3e of Table 3 the value of “r” in formula VII is 2.

In a specific embodiment, the monomer provided by the present invention has formula VIII. Those skilled in the art will recognize that the monomer having formula VIII is encompassed by both formula I and formula VII.

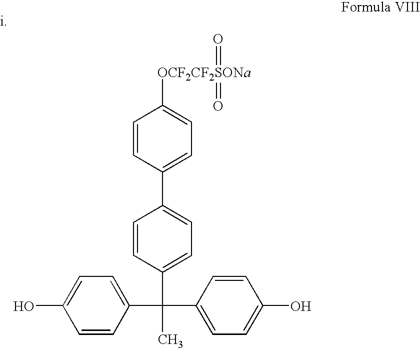

The monomers of the invention may be formed by reactions known to those skilled in the art. An exemplary reaction includes carbon-carbon bond formation via the Suzuki coupling reaction between a borate ester and, for example an aryl bromide catalyzed by a palladium catalyst. In a number of embodiments, known Suzuki coupling reaction methods and conditions are suitable for the preparation of the monomers provided by the present invention. Suitable reaction conditions may include the use of a polar aprotic reaction solvent at moderate temperatures. In one embodiment, the Suzuki coupling reaction is carried out at a temperature in a range from about ambient temperature to about 200° C. In another embodiment, the Suzuki coupling reaction is carried out at a temperature in a range from about 50° C. to about 150° C.

Other carbon-carbon bond forming reactions which may be employed in the preparation of the monomers of the present invention include condensation of a ketone with an excess of a phenolic compound in the presence of an acid to provide a bisphenol compound. Analogous chemistry, i.e. reaction of an aryl amine with a ketone in the presence of an acid, may in certain instances be used for the preparation of aromatic diamines which are structural analogs of bisphenols.

In various embodiments, the monomers of the present invention comprise functional groups requiring suitable protection so that they do not interfere with the reacting species during polymer synthesis. Thus, in certain embodiments, starting materials used in the preparation of the monomers, synthetic intermediates used in the preparation of the monomers and/or the polymers, or the monomers used to prepare the polymers themselves comprising suitable protecting groups are employed. Protecting groups for functional groups are known in the art, and are given in, for example, Greene and Wuts, “Protective Groups on Organic Synthesis”, Third Edition, 1999.

As noted, the present invention provides novel monomers comprising functional groups T. The functional groups T are selected from the group consisting of hydroxyl groups, amine groups, carboxylic acid groups, carboxylic acid ester groups, and thiol groups. Reactions of functional groups T with functional groups on comonomers having complementary reactivity to the functional groups T are well known in the art, and may be used here to make polymers. In one embodiment, T is a hydroxyl group and may be reacted with a carboxylic acid or a carboxylic acid ester or a carboxylic acid anhydride or a carboxylic acid chloride to form a polyester. In an alternate embodiment, T is a hydroxyl group which is converted to the corresponding salt and then reacted with a comonomer comprising a reactive aryl halide to form a polyether. In another embodiment, T is an amine which may be reacted with a carboxylic acid or a carboxylic acid ester or a carboxylate acid anhydride to form a polyamide. In yet another embodiment, T is a primary amine (—NH₂) which may be reacted with a cyclic carboxylic anhydride to form a polyimide. In yet still another embodiment, T is a thiol group which may be used to make, for example, a polythioester, or a polythioether. In another embodiment, T is a carboxylic acid ester which may be reacted with a comonomer comprising reactive hydroxyl groups to afford a polyester.

The monomers provided by the present invention are useful in the preparation of polymers comprising superacidic functional groups. In one embodiment, the monomer comprises two T groups, both of which are hydroxyl groups. The monomer is thus a dihydroxy aromatic compound and may be converted to a polymer, for example a polycarbonate, a copolycarbonate, a polyarylate, a copolyarylate, a copolyestercarbonate, a polyether, a polyether sulfone, or a polyether imide, by means of such hydroxyl groups. For example, where the monomer is a dihydroxy aromatic compound, for example Entry-1a of Table 1, the monomer may be polymerized under interfacial conditions with phosgene to provide a homopolycarbonate comprising structural units derived from said monomer. Interfacial conditions are illustrated by reactions commonly employed to make bisphenol A polycarbonate, namely reaction at or near ambient temperature of a dihydroxy aromatic compound with phosgene in a mixture of water and a water immiscible solvent such as methylene chloride in the presence of a water soluble base (e.g. sodium hydroxide) and a phase transfer catalyst such as triethylamine. In one embodiment, the present invention provides a monomer selected from the group consisting of monomers having formula I, monomers having formula V, and monomers having formula VII, which may be converted to a polymer by reaction under interfacial conditions with a comonomer (for example a bisphenol such as bisphenol A) to provide a copolycarbonate comprising structural units derived from a monomer comprising a superacidic functional group and structural units derived from the comonomer. In an alternate embodiment, the present invention provides a monomer selected from the group consisting of monomers having formula I, monomers having formula V, and monomers having formula VII, which may be reacted under melt polymerization conditions with a diaryl carbonate to afford a melt polycarbonate. Melt polymerization conditions are illustrated by the polymerization reaction conditions typically employed when reacting a bisphenol (e.g. bisphenol A) with a diaryl carbonate (e.g. diphenyl carbonate) in the presence of a minute amount of a basic catalyst such as sodium hydroxide at a temperature in a range between about 150 and 300° C. at subatmospheric pressure. In yet another embodiment, the present invention provides a monomer selected from the group consisting of monomers having formula I, monomers having formula V, and monomers having formula VII, which may be reacted under interfacial conditions with a bishaloformate (e.g. bisphenol A bischloroformate) to provide a polycarbonate comprising structural units derived from said monomer.

In another embodiment, the monomer provided by the present invention comprises hydroxyl groups which may be used to prepare a polyester. For example, the monomer may be reacted with a comonomer which is a carboxylate ester, a carboxylic anhydride, or a carboxylic acid halide under melt or interfacial polymerization conditions as appropriate to afford a polyester.

In one embodiment, the monomer provided by the present invention may be used in the preparation of polyether sulfones. Thus, for example, the disodium salt of the monomer of Entry-1d of Table 1 together with the disodium salt of bisphenol A may be reacted with bis(4-chlorophenyl)sulfone in orthodichlorobenzene at a temperature between about 100 and about 250° C. in the presence of a phase transfer catalyst such as hexaethyl guanidinium chloride to provide a product polyethersulfone. The product polyethersulfone may be used in polymer electrolyte membrane applications.

As will be appreciated by those skilled in the art, the monomers provided by the present invention may be used to make a wide variety of polymer compositions which may be useful in many different applications, for example, membranes. As noted, monomers comprising aromatic hydroxyl groups (i.e. a hydroxy group attached to an sp² carbon atom of an aromatic ring) may be used in the preparation of polycarbonates, polyesters, and polyethersulfones to name a few. Amine substituted monomers provided by the present invention, such as Entry-2b of Table 2, may be employed in the preparation of polyamides, polyimides, polyether imides, and the like. For example, monomer of Entry-2b of Table 2 and m-phenylene diamine may be condensed with bisphenol A dianhydride (BPADA) in orthodichlorobenzene at a temperature in a range between about 100 and about 220° C. in the presence of a slightly basic catalyst such as sodium phenyl phosphite to provide a polyether imide comprising structural units derived from the monomer of Entry-2b.

Reaction conditions useful for the preparation of polymer compositions comprising structural units derived from the monomers provided by the present invention include the use of polar solvents and bases of suitable strength. Exemplary solvents include chloroform, methylene chloride, orthodichlorobenzene, veratrole, anisole, the like, and combinations thereof. Exemplary bases include triethylamine, sodium hydroxide, potassium hydroxide, and the like, and combinations thereof. Suitable catalysts may also be employed to effect the polymerization reaction.

In certain embodiments, the polymerization reaction may be conducted at a suitable temperature that ranges from about room temperature to about the boiling point of the solvent of choice. The polymerization may also be conducted at atmospheric pressure, subatmospheric pressures, or superatmospheric pressures. The polymerization reaction is conducted for a time period necessary to achieve polymer of a suitable molecular weight. The molecular weight of a polymer is determined by any of the techniques known to those skilled in the art, and include viscosity measurements, light scattering, osmometry, and the like. The molecular weight of a polymer is typically represented as a number average molecular weight Mₙ, or weight average molecular weight, M_{w}. A particularly useful technique to determine molecular weight averages is gel permeation chromatography (GPC), from wherein both number average and weight average molecular weights are obtained. In some embodiments, polymers of M_{w} greater than 30,000 grams per mole (g/mol) is desirable, in other embodiments, polymers of M_{w} greater than 50,000 g/mol is desirable, while in yet other embodiments, polymer of M_{w} greater than 80,000 g/mol is desirable.

The polymerization reaction may be controlled the addition of a suitable monofunctional reactant, sometimes also referred to in the art as “end-capping agents”, or “chain stoppers”. The chain stopper serves to limit polymer molecular weight. Suitable phenolic chain stoppers include phenol, p-cumylphenol, and the like. Suitable aromatic amine chain stoppers include aniline, 2,4-dimethylaniline, and the like. Suitable aromatic halide chain stoppers include, 4-chlorophenyl phenyl sulfone, 4-fluorophenyl phenyl sulfone, 4-chlorophenyl phenyl ketone, and the like.

The polymers prepared using the monomers provided by the present invention may be isolated and purified by techniques known in the art. Techniques to be used depend on the choice of solvents, monomers, and catalysts. In one embodiment, the product mixture is obtained as a solution comprising the product polymer, residual monomers, by-products, and catalyst. This solution may be added dropwise into a solvent which dissolves residual monomers, by-products, and catalyst from the polymerization reaction, but in which the product polymer is insoluble. Such solvents may also be referred to as a nonsolvent for the polymer, or simply as a nonsolvent. Subsequently, the polymer may be isolated by solid separation techniques known in the art, which include filtration, Mott filtration, centrifugation, decantation, and the like, and combinations thereof. The isolated polymer may then be dissolved in a solvent and precipitated out of a nonsolvent as many times as deemed necessary by the practitioner to obtain a desired level of polymer purity. The polymer may be dried under vacuum, with or without the application of heat to dry any trace solvents and/or nonsolvents associated with it.

In some embodiments, the polymer is obtained from the one or more purification steps as a solution which may be used in further applications, for example in the preparation of a cast film. Polymer films may be obtained by casting the polymer solution onto a suitable substrate and allowing the solvent to evaporate. Subsequently, depending on the application, the film may be removed from the substrate, or may be used in combination with the substrate. In certain embodiments films are prepared by spin casting a solution of the product polymer onto a suitable substrate.

In particular embodiments, the polymer is first isolated as a solid and then melt extruded to provide a stand alone film. In other embodiments, the solid polymer may be compression molded at suitable temperatures and pressures to obtain a film of desired thickness. Other techniques for film formation are known in the art, and may be used here.

In one embodiment, the present invention provides a monomer which may be used to make polymers useful in solid polymer electrolyte membrane fuel cell applications. It has been found that the superacidic groups present in the polymers derived from the monomers provided by the present invention exhibit higher conductivities (i.e., >0.1 S/cm) than polymers having aromatic sulfonic acid groups at the same effective concentrations.

In one embodiment, the monomer provided by the present invention may be used to prepare polymers useful in proton exchange membranes. Proton exchange membranes are important components of fuel cell devices. A fuel cell device transforms the chemical energy liberated during the electrochemical reaction of hydrogen and oxygen to electrical energy. An exemplary proton exchange membrane-containing fuel cell comprises a membrane electrode assembly (MEA), which comprises at least one electrode, each electrode comprising an anode side, a cathode side, and a proton exchange membrane that separates the anode side from the cathode side. A stream of hydrogen is delivered to the anode side of the membrane-electrode assembly. At the anode side, the hydrogen is converted catalytically into protons and electrons. This oxidation reaction may be represented by: H₂→2H⁺+2e⁻. The protons formed permeate through the proton exchange membrane to the cathode side. The electrons, in turn, travel along an external load circuit to the cathode side of the MEA, thus creating the current output of the fuel cell. Meanwhile, a stream of oxygen is delivered to the cathode side of the MEA. At the cathode side, oxygen molecules react with the protons permeating through the polymer electrolyte membrane and the electrons arriving through the external circuit to form water molecules. This reduction reaction is represented by: 4H⁺+4e⁻+O₂→2H₂O. Typically, the polymer composition used as the membrane must possess barrier properties such that gases may not pass from one side of the cell to the other side of the cell, a problem known in the art as gas crossover. Further, the polymer membrane must be resistant to the harsh chemical environments at the anode and the cathode. The polymers provided by the present invention are useful as in proton exchange membranes, and effect the efficient transport/permeation of protons from the anode side of the MEA to the cathode side of the MEA, thus effecting efficient conversion of chemical energy to electrical energy. Fuel cells such as those described herein find use in transport applications such as automobiles, portable applications such as mobile phones, stationary applications such as domestic electricity, and the like.

The monomers provided by the present invention may be used to prepare polymers used in polymer compositions comprising additives which may improve the polymer composition properties, such as mechanical properties, aesthetic properties, and the like, for example. Exemplary additives include, but are not limited to, additives which improve scratch resistance, hardeners, colorants, fillers, hardeners, and so on, and combinations thereof.

Without further elaboration, it is believed that one skilled in the art can, using the description herein, utilize the present invention to its fullest extent. The following examples are included to provide additional guidance to those skilled in the art in practicing the claimed invention. The examples provided are merely representative of the work that contributes to the teaching of the present application. Accordingly, these examples are not intended to limit the invention, as defined in the appended claims, in any manner.

### EXAMPLES

General Procedures: Tetrahydrofuran, toluene, and NMP were purified through a Solv-Tek solvent purification system, containing columns packed with activated R3-15 deoxygenation catalyst and 8-14 mesh activated alumina. (Solv-Tek, Inc. 216 Lewisville Road Berryville, Va. 22611). Pd(PPh₃)₄ was purchased from Strem Chemicals, Newburyport, Mass., and used as received. 2-(4-Bromophenoxy)tetrafluoroethanesulfinate and 2-(4-bromophenoxy)tetrafluoroethanesulfonyl fluoride were synthesized according to the procedure given in Feiring et al., J. Fluor. Chem., Volume 105, pp. 129-135 (2000). 5-Bromoresorcinol was synthesized according to the procedure given in Dol, et al., Eur. J. Org. Chem. pp. 359-364 (1998). All other chemicals were purchased from Aldrich Chemical Company, Milwaukee, Wis. and used as received, unless otherwise noted. All reactions with air- and/or water-sensitive compounds were carried out under dry nitrogen (purified through Trigon Technologies Big Moisture Traps, Trigon Technolgies, Rancho Cordova, Calif.) using standard Schlenk line techniques. NMR spectra were recorded on a Bruker Advance 400 (¹H, 400 MHz and ¹³C, 100 MHz) spectrometer and referenced versus residual solvent shifts. Molecular weights are reported here as number average (Mₙ) or weight average (M_{w}) molecular weight and were determined by gel permeation chromatography (GPC) analysis on a Perkin Elmer Series 200 instrument equipped with RI detector. Polyethyleneoxide molecular weight standards were used to construct a broad standard calibration curve against which polymer molecular weights were determined. The temperature of the gel permeation column (Polymer Laboratories PLgel 5 μm MIXED-C, 300×7.5 millimeter (mm)) was 40° C. and the mobile phase was 0.05 Molar (M) LiBr in DMAc. Polymer thermal analysis was performed on a Perkin Elmer DSC7 equipped with a TAC7/DX thermal analyzer and processed using Pyris Software. Glass transition temperatures were recorded on the second heating scan.

### Example 1

### Preparation of Protected 5-Bromoresorcinol (4)

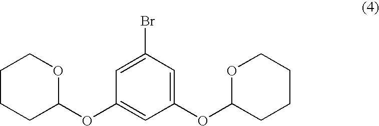

5-Bromoresorcinol (6.89 grams (g), 36.5 millimoles (mmol)) and pyridinium p-toluenesulfonate (0.14 g, 0.56 mmol) were added with chloroform (CHCl₃) to a 500 milliliters (ml) round-bottomed flask. While stirring, 3,4-dihydro-2H-pyran (10.0 ml, 110 mmol) was added dropwise over 30 minutes (mins). After an additional 30 minutes, all solids were dissolved in solution. Spot Thin Layer Chromatography (TLC) showed full conversion to product. 2 Molar (M) NaOH (18 ml, 36 mmol) was added and the biphasic mixture was stirred vigorously for 1 hour. The yellow organic layer was collected and the aqueous layer was washed with chloroform (3×30 ml). The combined organic layers were washed with water (1×100 ml) and brine (1×100 ml), dried over MgSO₄, filtered, and dried in vacuo to leave a dark yellow oil. The product was precipitated as an off-white solid by dissolving the oil in a minimal amount of ethanol (10 ml) and adding a 1:1 solution (50 ml) of acetonitrile:water to give 10.8 g of product at 83% yield. ¹H NMR spectrum was in agreement with the assigned structure of compound (4).

### Example 2

### Preparation of Boronate Ester (5)

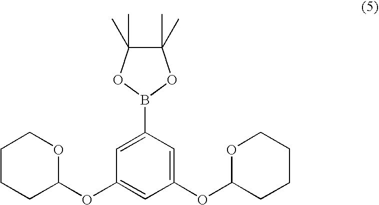

Magnesium turnings were activated by washing with 15% HCl_{(aq)} (v/v) followed by washing with water, then acetone, and drying in vacuo. Under nitrogen atmosphere, compound (4) (1.45 g, 4.06 mmol), magnesium turnings (0.285 g, 11.7 mmol), THF (10 ml), and 2-isopropoxy-4-4,4,5,5-tetramethyl-1,3,2-dioxaborolane (1.10 ml, 5.39 mmol) were added to an oven-dried, 100 ml, three-necked round bottom flask equipped with a thermocouple and refluxing condenser. Under nitrogen, 1,2-dibromoethane (0.10 ml, 1.16 mmol) was added via syringe to the stirring mixture at room temperature. After approximately 5 minutes, the reaction initiated and the temperature rose. Note: Grignard reactions are highly exothermic, and appropriate precautions should be taken. The reaction was stirred for 4 hours and then CH₂Cl₂ (50 ml) and water (50 ml) were added. The biphasic mixture was filtered, the organic layer collected and the aqueous layer was washed with CH₂Cl₂ (3×25 ml). The combined organic layers were washed with brine (1×75 ml), dried over MgSO₄, filtered, and dried in vacuo to leave a light yellow oil that crystallized over the course of an hour. Cold methanol was added and the white solid was collected by filtration and washed with cold methanol to give 1.02 g of product at 62% yield. ¹H NMR (CDCl₃, 400 MHz) δ 7.15 (2H, t, J=2.0 Hz, ArH), 6.91 (1H, quartet, J=2.4 Hz, ArH), 5.51 (2H, m, CH), 3.93 (2H, m, CHₐH_{b}O), 3.77 (2H, m, CHₐH_{b}O), 1.6-2.1 (12H, bm, CH₂), 1.34 (12H, s, CH₃).

### Example 3

### Preparation of Sulfonyl Chloride (6)

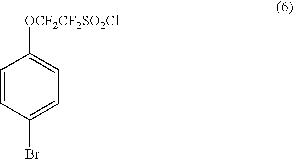

2-(4-Bromophenoxy)tetrafluoroethanesulfinate (4.40 g, 12.4 mmol) was dissolved in deionized water. Bleach (a 6.15% w/v aqueous solution of sodium hypochlorite, 40 ml) was added at room temperature, resulting in a cloudy suspension. The mixture was vigorously stirred for 2 minutes. The organics were extracted with ether (4×50 ml). The combined organic layers were washed with brine (2×50 ml), dried over MgSO₄, filtered, and dried in vacuo to leave the 4.33 g of product as a colorless liquid at 95% yield. ¹H NMR (CDCl₃, 400 MHz) δ 7.57 (2H, d, J=8.8 Hz, ArH), 7.14 (2H, d, J=8.8 Hz, ArH).

### Example 4

### Preparation of Sulfonate Ester (7)

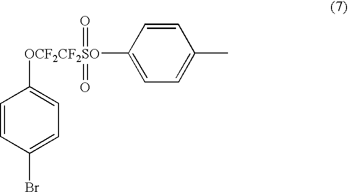

In an oven-dried, 100 ml round bottomed flask, sodium p-cresolate (1.61 g, 12.4 mmol) was dissolved in 70 ml DMSO:acetonitrile (1:1) and cooled to 0° C. in an ice bath. 2-(4-Bromophenoxy)tetrafluoroethanesulfonyl fluoride (4.10 g, 11.5 mmol) was added dropwise over the course of 30 minutes. The solution was allowed to stir at 0° C. for 2 hours and then gradually warmed to room temperature and stirred for 24 hours. Acetonitrile was removed in vacuo and water was added (100 ml). The organic products were extracted with ether (4×50 ml). The combined organic layers were combined, washed with 1 M NaOH (2×50 ml) to remove unreacted cresol, washed with brine (2×50 ml), dried over MgSO₄, filtered, and dried in vacuo. The product was purified by fractional vacuum distillation (20 mmHg, 125-130° C.) to give 2.92 g of a colorless liquid at 57% yield. ¹H NMR (CDCl₃, 400 MHz) δ 7.54 (2H, d, J=8.6 Hz, ArH), 7.23 (4H, m, ArH), 7.15 (2H, d, J=9.2 Hz, ArH), 2.40 (3H, s, CH₃).

### Example 5

### Preparation of Monomer (1j)

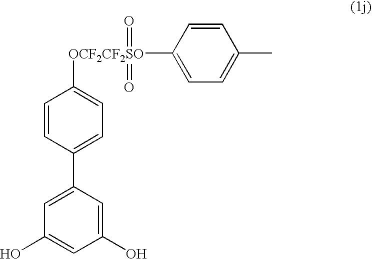

In an oven-dried Schlenk tube, Compound (5) (0.782 g, 1.93 mmol), Compound (7) (0.658 g, 1.48 mmol), Pd(PPh₃)₄ (0.083 g, 0.072 mmol), and Cs₂CO₃ (0.975 g, 2.99 mmol) were added. The flask was evacuated and DMF (5 ml) was added via syringe under nitrogen atmosphere. The flask was slightly evacuated to remove the headspace, and the reaction was stirred vigorously at 100° C. for 24 hours. The mixture was cooled to room temperature, water (50 ml) was added, and diethyl ether (4×50 ml) was used to extract the crude material. The organic fractions were combined, washed with brine (2×50 ml), dried over MgSO₄, filtered, and dried in vacuo. Silica gel chromatography was used to purify the product compound (gradient elution: 5% to 10% to 20% EtOAc/hexane). The colorless oil was dissolved in THF (10 ml) and MeOH (2 ml) and concentrated HCl (2 drops) was added. The light yellow solution was stirred for 1 hr. Saturated aqueous sodium bicarbonate solution (10 ml) was added, the organics were extracted with ether (3×50 ml), the combined organic fractions were washed with brine (2×50 ml), dried over MgSO₄, filtered, and dried in vacuo to leave 0.53 g of a light yellow oil that partially crystallized overnight at 77% yield. ¹H NMR (CDCl₃, 400 MHz) δ 7.55 (2H, d, J=8.8 Hz, ArH), 7.30 (2H, d, J=8.8 Hz, Hz, ArH), 7.24 (4H, bs, ArH), 6.62 (2H, d, J=2.0 Hz, ArH), 6.38 (2H, t, J=2.0 Hz, ArH), 5.10 (2H, s, OH), 2.40 (3H, s, CH₃).

### Example 6

### Preparation of Bisphenol (8)

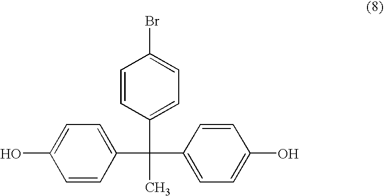

In a 500 ml round-bottom flask, 4-bromoacetophenone (47.0 g, 0.236 mol), phenol (139.4 g, 1.471 mol), and 75% H₂SO_{4(aq)} (75 ml) were stirred at 50° C. for 2.5 days. The solution turned a dark red over the course of the reaction. The organics were extracted with diethyl ether (4×200 ml). The combined organic layers were washed with saturated sodium bicarbonate (2×500 ml), dried over MgSO₄, filtered, and dried in vacuo to leave a viscous, yellow oil. Gradient silica-gel column chromatography (5% to 50% ethyl acetate/hexane) was performed to separate the unreacted phenol and 4-bromoacetophenone from the desired product. After crystallization from a 1:4 solution of toluene:heptane (400 ml) at −20° C., 39.9 g of product was obtained in 46% yield. ¹H NMR (CDCl₃, 400 MHz) δ 7.39 (2H, d, J=8.8 Hz, Br—ArH), 6.98 (2H, d, J=8.4 Hz, Br—ArH), 6.95 (4H, d, J=8.8 Hz, OH—ArH), 6.75 (4H, d, J=8.8 Hz, OH—ArH), 4.78 (2H, s, OH), 2.11 (3H, s, CH₃).

### Example 7

### Preparation of Protected Bisphenol (9)

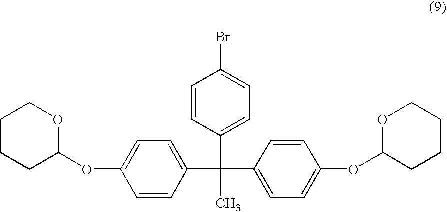

Bisphenol (8) (5.49 g, 14.9 mmol) and pyridinium p-toluenesulfonate (0.120 g, 0.477 mmol) were treated in chloroform (150 ml) with 3,4-dihydro-2H-pyran (10.0 ml, 110 mmol) as in Example 1 to provide protected bisphenol (9) (7.75 g, 97% yield). ¹H NMR (CDCl₃, 400 MHz) δ 7.38 (2H, d, J=8.8 Hz, Br—ArH), 6.97 (10H, m, ArH), 5.41 (2H, t, J=3.2 Hz, CH), 3.95 (2H, m, CHₐH_{b}O), 3.62 (2H, m, CHₐH_{b}O), 2.11 (3H, s, CH₃), 1.5-2.1 (12H, bm, CH₂).

### Example 8

### Preparation of Boronate Ester (10)

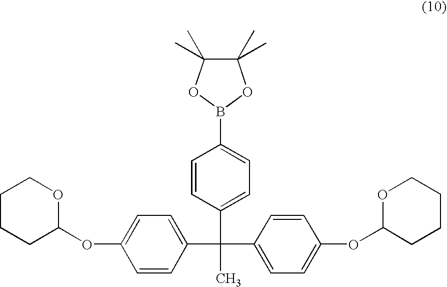

Protected bisphenol (9) (28.1 g, 52.3 mmol) was dissolved in THF (200 ml) in an oven-dried 500 ml round-bottom flask. The solution was cooled to −78° C. and n-butyl lithium (22.0 ml, 55.0 mmol, 2.5 M in hexane) was added slowly via syringe. The solution was allowed to slowly warm to −30° C. and stirred for an additional 15 minutes. The yellow solution was again cooled to −78° C. and 2-isopropoxy-4-4,4,5,5-tetramethyl-1,3,2-dioxaborolane (12.5 ml, 61.3 mmol) was added via syringe. The solution was allowed to warm to room temperature and was stirred overnight, after which time a white precipitate was observed. Methylene chloride (CH₂Cl₂) (300 ml) and water (300 ml) were added and the organic layer was collected. The aqueous layer was washed with CH₂Cl₂ (3×100 ml) and the combined organic layers were washed with brine (2×150 ml), dried over MgSO₄, filtered, and dried in vacuo to afford the crude product as a white solid which was triturated with cold methanol, filtered and washed with cold methanol to afford boronate ester (10) (26.2 g) in 86% yield. ¹H NMR (CDCl₃, 400 MHz) δ 7.72 (2H, d, J=8.4 Hz, Br—ArH), 7.14 (2H, d, J=8.4 Hz, Br—ArH), 7.00 (4H, d, J=8.8 Hz, O—ArH), 6.94 (4H, d, J=8.8 Hz, O—ArH), 5.40 (2H, t, J=3.2 Hz, CH), 3.95 (2H, m, CHₐH_{b}O), 3.61 (2H, m, CHₐH_{b}O), 2.14 (3H, s, CH₃), 1.6-2.1 (12H, bm, CH₂), 1.35 (12H, s, CH₃).

### Example 9

### Preparation Of 4-t-Butylphenyl Sulfonate (11)

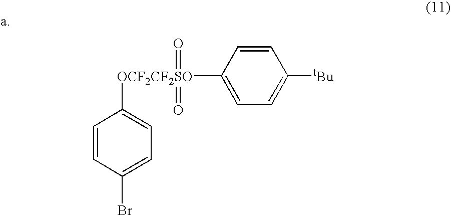

An oven-dried, 250 ml round-bottom flask was charged with 4-tert-butylphenol (8.58 g, 57.1 mmol), triethylamine (5.91 g, 59.6 mmol), and acetonitrile (25 ml) and cooled to −30° C. A solution of 2-(4-Bromophenoxy)tetrafluoroethanesulfonyl fluoride (4.10 g, 11.5 mmol) in acetonitrile (25 ml) was then added at −30° C. via cannula over the course of about 30 minutes. The reaction mixture was allowed to warm to 0° C. and then stirred for 6 hours at 0° C. The resultant colorless solution was then gradually warmed to room temperature and stirred for 16 hours. Acetonitrile was removed in vacuo and water was added (100 ml). The organic products were extracted with diethyl ether (4×100 ml). The organic layers were combined, washed with 0.05 M NaOH (2×100 ml) to remove unreacted 4-tert-butylphenol, washed with brine (2×100 ml), dried over MgSO₄, filtered, and dried in vacuo. The product was purified by silica-gel column chromatography using 5% ethyl acetate/hexane as eluent to give 24.9 g of a colorless liquid at 96% yield. ¹H NMR (CDCl₃, 400 MHz) δ 7.54 (2H, d, J=8.4 Hz, ArH), 7.46 (2H, d, J=9.2 Hz, ArH), 7.25 (2H, d, J=9.2 Hz, ArH), 7.14 (2H, d, J=8.8 Hz, ArH), 1.34 (9H, s, CH₃).

### Example 10

### Preparation of Protected Monomer (12)

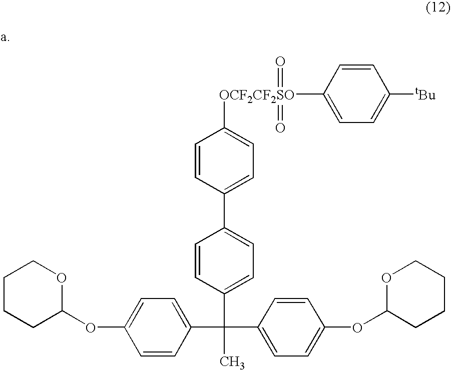

To an oven-dried 500 ml round-bottom flask was charged boronate ester (10) (10.5 g, 17.9 mmol), 4-t-butylphenyl sulfonate (11) (7.05 g, 14.5 mmol), Pd(PPh₃)₄ (0.836 g, 0.072 mmol), and Cs₂CO₃ (7.74 g, 29.9 mmol). The atmosphere in the flask was exchanged by evacuation and introduction of nitrogen gas. DMF (50 ml) was added via syringe under a nitrogen atmosphere. The flask was evacuated slightly to remove remaining unwanted headspace gases, and the reaction mixture was stirred vigorously at 80° C. for 24 hours. The reaction mixture was then cooled to room temperature, and water (400 ml) and CH₂Cl₂ (400 ml) were added. The resulting milky suspension was filtered through Celite on a C-frit filter. The aqueous phase was extracted with CH₂Cl₂ (5×100 ml). The combined organic fractions were washed with brine (2×300 ml), dried over MgSO₄, filtered, and evaporated in vacuo to afford a light yellow oil. 10% Ethyl acetate/hexanes (50 ml) and methanol (100 ml) were added to solubilize the oil. White crystals started forming within 30 minutes and the flask was placed in a freezer (−20° C.) overnight to give 9.45 g of the heterocoupled product in 75% yield. ¹H NMR (CDCl₃, 400 MHz) δ 7.61 (2H, d, J=8.8 Hz, ArH), 7.46 (4H, m, ArH), 7.29 (4H, m, ArH), 7.20 (2H, d, J=8.0 Hz, ArH), 7.05 (4H, d, J=9.2 Hz, ArH), 6.97 (4H, d, J=8.8 Hz, ArH), 5.42 (2H, t, J=3.2 Hz, CH), 3.96 (2H, m, CHₐH_{b}O), 3.62 (2H, m, CHₐH_{b}O), 2.18 (3H, s, CH₃), 1.6-2.1 (12H, bm, CH₂), 1.35 (9H, s, CH₃).

### Example 11

### Preparation of Monomer (13)

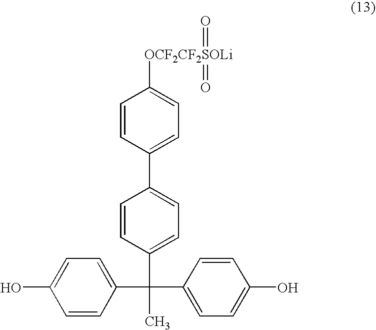

Protected monomer (12) (8.05 g, 9.33 mmol) was dissolved in THF (80 ml) and MeOH (20 ml). Concentrated HCl (25 drops) was added via syringe and the yellow solution was stirred at room temperature for 2 hours. Lithium hydroxide (8.00 g, 334 mmol) was dissolved in water (100 ml) and added to the yellow solution. The solution was stirred vigorously at 80° C. for 5 hours, and then cooled to room temperature. The basic solution was neutralized with HCl to pH 8, and then the volatiles were removed in vacuo to leave a brown oil. Ethyl acetate (100 ml) and brine (100 ml) were added and the organic layer was collected. The brine layer was washed with ethyl acetate (2×100 ml). The combined organic layers were washed with brine (1×100 ml), dried over MgSO₄, filtered, and evaporated under reduced pressure to afford a white solid. The solid was triturated with hot CHCl₃ for 5 minutes, filtered, washed with additional hot CHCl₃ and dried under vacuum overnight at 80° C. ¹H NMR (DMSO-d₆, 400 MHz) δ 9.28 (2H, s, OH), 7.73 (2H, d, J=8.8 Hz, ArH), 7.58 (2H, d, J=8.4 Hz, ArH), 7.29 (2H, d, J=8.4 Hz, ArH), 7.11 (2H, d, J=8.4 Hz, ArH), 6.85 (4H, d, J=8.4 Hz, ArH), 6.67 (4H, d, J=8.4 Hz, ArH), 2.05 (3H, s, CH₃). The ¹⁹F NMR spectra was also in agreement with the assigned structure of monomer (13).

### Example 12

### Synthesis of Monomer (14)

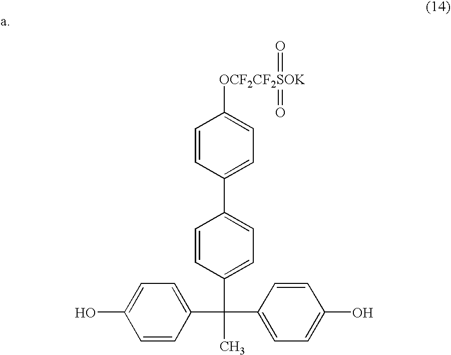

Boronate ester (10) (15.6 g, 26.7 mmol) and 4-t-butylphenyl sulfonate (11) (10.8 g, 22.3 mmol) were coupled as in Example 10 to afford protected monomer (12). The white solid was then dissolved in THF (70 ml) and MeOH (30 ml). Concentrated HCl (0.2 ml) was added via syringe and the yellow solution was stirred at room temperature for 2 hours. Potassium hydroxide (12.8 g, 228 mmol) was dissolved in water (25 ml) and added to the yellow solution. The solution was stirred vigorously at 80° C. for 36 hours, and then cooled to room temperature. The basic solution was neutralized with HCl to pH 8, and then the volatiles were removed in vacuo to afford a brown oil. The product was purified and recovered as in Example 11 to give monomer (14) as a white solid (9.56 g) in 72% yield. ¹H NMR (DMSO-d₆, 400 MHz) δ 9.29 (2H, s, OH), 7.73 (2H, d, J=8.8 Hz, ArH), 7.58 (2H, d, J=8.4 Hz, ArH), 7.30 (2H, d, J=8.0 Hz, ArH), 7.12 (2H, d, J=8.4 Hz, ArH), 6.86 (4H, d, J=8.8 Hz, ArH), 6.68 (4H, d, J=8.4 Hz, ArH), 2.05 (3H, s, CH₃). ¹⁹F NMR (CDCl₃, 564.4 MHz) δ −76.5 (2F), −112.4 (2F).

### Example 13

### Polyethersulfone Comprising Structural Units Derived from Monomer (14)

Polymerization was carried out in an oven-dried round bottom flask equipped with a mechanical stirrer, an addition funnel, and a simple distillation apparatus. Monomer (14) (2.275 g, 3.800 mmol), 4,4′-difluorodiphenylsulfone (DFDPS) (0.911 g, 3.58 mmol), and K₂CO₃ (2.02 g, 14.6 mmol) were added to the reaction flask and DMSO (10.0 ml) and toluene (5.0 ml) were added via syringe. Under a nitrogen atmosphere, the mixture was stirred at 150° C. for 6 hours with azeotropic water removal. Then, biphenol (0.343 g, 1.84 mmol) and 4,4′-difluorodiphenylsulfone (DFDPS) (0.524 g, 2.063 mmol), were added, along with DMSO (5 ml) and toluene (2 ml). The polymerization reaction mixture was stirred under a nitrogen atmosphere at 150° C. for 4.75 hours. The polymerization reaction mixture was sampled and assayed by GPC. The weight average and number average molecular weights M_{w} and Mₙ were found to be 125,000 grams per mole and 30,700 grams per mole, respectively. The polymer was precipitated into vigorously stirred isopropanol (400 ml), filtered, washed with methanol and water, and dried in vacuo at 100° C. overnight.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A monomer having formula I 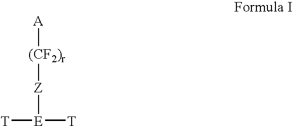
wherein E is a C₅-C₅₀ aromatic radical;
Z is a bond, O, S, SO, SO₂, a C₁-C₂₀ aliphatic radical, a C₃-C₄₀ aromatic radical, or a C₄-C₂₀ cycloaliphatic radical;
“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;
T is a functional group selected from the group consisting of hydroxyl, amine, carboxylic acid, carboxylic acid ester, and thiol; and
“r” is an integer ranging from 1 to 20.

2. The monomer of claim 1, wherein T is a hydroxyl group.

3. The monomer of claim 1, wherein T is an amine group.

4. The monomer of claim 1, wherein Z is an oxygen.

5. The monomer of claim 1, wherein “r” is 2.

6. The monomer of claim 1, wherein Z is a carbonyl group.

7. The monomer of claim 1, wherein E is a C₆ aromatic radical having formula II. 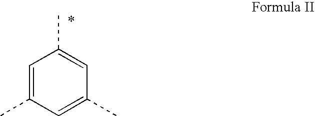
wherein the dashed line -----* indicates a point of attachment of the group -Z(CF₂)ᵣA and the dashed lines ------ indicate a point of attachment of the groups T.

8. The monomer of claim 1, wherein E is a C14 aromatic radical having formula II 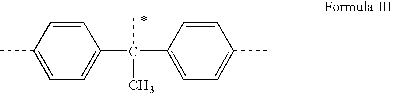
wherein the dashed line [symbol] indicates a point of attachment of the group -Z(CF₂)ᵣA and the dashed lines [symbol] indicate a point of attachment to the groups T.

9. The monomer of claim 1, wherein “A” is a salt of a sulfonic acid moiety, said salt having formula SO₃M, wherein M is selected from the group consisting of potassium, sodium, lithium, and cesium.

10. The monomer of claim 1, wherein E comprises a perfluorinated C₁-C₂₀ aliphatic radical, or a perfluorinated C₃-C₂₀ aromatic radical.

11. The monomer of claim 10, wherein E comprises a perfluorinated C₁-C₂₀ aliphatic radical.

12. A monomer having formula V 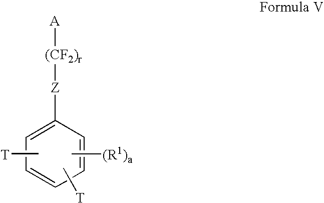
wherein Z is a bond, O, S, SO, SO₂, a C₁-C₂₀ aliphatic radical, a C₃-C₄₀ aromatic radical, or a C₄-C₂₀ cycloaliphatic radical;
“A” is a sulfonate moiety selected from the group consisting of a sulfonic acid moiety, a salt of a sulfonic acid moiety having formula SO₃M wherein M is an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;
T is a functional group selected from the group consisting of hydroxyl, amine, carboxylic acid, carboxylic acid ester, and thiol;
R¹ is a C₁-C₄₀ aliphatic radical, a C₃-C₄₀ aromatic radical, or a C₄-C₂₀ cycloaliphatic radical;
“r” is an integer ranging from 1 to 20; and
“a” is 0 or an integer ranging from 1 to 3.

13. The monomer of claim 12, wherein T is a hydroxyl.

14. The monomer of claim 12, wherein T is an amine.

15. The monomer of claim 12, wherein r is 2.

16. The monomer of claim 12, wherein “A” is a salt of a sulfonic acid moiety, said salt having formula SO₃M, wherein M is selected from the group consisting of potassium, sodium, lithium, and cesium.

17. The monomer of claim 12, wherein “A” is a sulfonate ester.

18. The monomer of claim 12, having formula VI

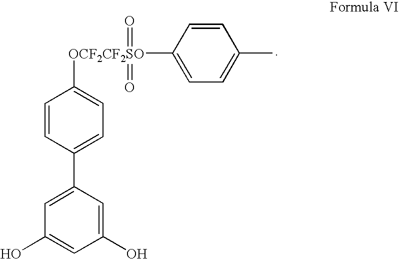

19. A monomer having formula VII 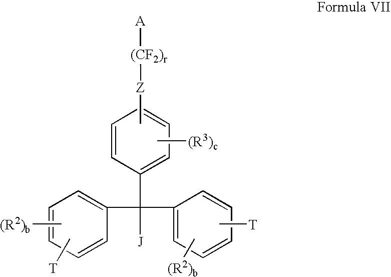
wherein J is a hydrogen, a C₁-C₂₀ aliphatic radical, a C₃-C₂₀ aromatic radical, or a C₄-C₂₀ cycloaliphatic radical;
Z is a bond, O, S, SO, SO₂, a C₁-C₂₀ aliphatic radical, a C₃-C₄₀ aromatic radical, or a C₄-C₂₀ cycloaliphatic radical;
“A” is a sulfonate moiety selected from the group consisting of a sulfonic acid moiety, a salt of a sulfonic acid moiety having formula SO₃M wherein M is a hydrogen, an inorganic cation, or an organic cation, and a sulfonate ester moiety having formula SO₃R, wherein R is a C₁-C₂₀ aliphatic radical, a C₃-C₂₀ aromatic radical, or a C₄-C₂₀ cycloaliphatic radical;
T is a functional group selected from the group consisting of hydroxyl, amine, carboxylic acid, carboxylic acid ester, and thiol;
R² and R³ are independently at each occurrence a C₁-C₂₀ aliphatic radical, a C₃-C₄₀ aromatic radical, or a C₄-C₂₀ cycloaliphatic radical;
“r” is an integer ranging from 1 to 20;
“b” is 0 or an integer ranging from 1 to 4; and
“c” is 0 or an integer ranging from 1 to 4.

20. The monomer of claim 19, wherein T is a hydroxyl.

21. The monomer of claim 19, wherein T is an amine.

22. The monomer of claim 19, wherein J is a C₁-C₂₀ perfluorinated aliphatic radical, or a perfluorinated C₃-C₂₀ aromatic radical.

23. The monomer of claim 22, wherein J is a perfluorinated C₁-C₂₀ aliphatic radical.

24. The monomer of claim 19, wherein “r” is 2.

25. The monomer of claim 19, wherein A is a salt of a sulfonic acid moiety, said salt having formula SO₃M, wherein M is selected from the group consisting of potassium, sodium, lithium, and cesium.
